(19) United States
(12) Patent Application Publication
(10) Pub. No.: US 2019/0205002 A1
(43) Pub. Date: Jul. 4, 2019

(54) Continuous Improvement Tool

(71) Applicant: Talis Clinical LLC, Streetsboro, OH (US)

(72) Inventors: Gary Colister, Hudson, OH (US); Bishoy Magdalla, Hudson, OH (US); Giuseppe Saracino, Gainsville, FL (US); William Murphy, Aurora, OH (US); Harish Lecamwasam, Scottsdale, AZ (US)

(21) Appl. No.: 16/239,023

(22) Filed: Jan. 3, 2019

Related U.S. Application Data
(60) Provisional application No. 62/613,218, filed on Jan. 3, 2018.

(51) Int. Cl. G06F 3/0484; A61B 5/00; G06F 3/0482; G16H 10/60

(52) U.S. Cl. CPC G06F 3/0484; G16H 10/60; G06F 3/0482; A61B 5/7465

(57) ABSTRACT

A system for improving patient outcomes, including a telecommunications network; monitoring equipment for receiving and sending data via the network, and instructions for receiving data and accessing patient medical conditions; a patient database; a communication hub with instructions for collecting data and transmitting data to electronic devices and for storing data; a data storage engine including means for collecting data from electronic devices and instructions for transmitting data to any number of electronic devices; the telecommunication network providing access to all data, including continuous wave form, collected during treatment and quality data not included in any patient record storage location; and a user interface rules engine that provides the user with ability to select any point in time during the patient treatment to review details and create guidance rules so that data can be acquired, consolidated and actionable insights can be delivered in near-real time.

### BACKGROUND OF THE INVENTION

The present invention is directed to a continuous improvement tool, which facilitates monitoring patient vital signs and histories to continuously improve decisions regarding patient care.

The base foundation of any process control used to drive a continuous spiral of improvements is to monitor the process to ensure consistent compliance with the defined protocol. With knowledge that the protocol is consistently followed, data analysis may be used to assess the capabilities of the protocol and its ability to deliver the expected outcomes. There is any number of reasons for using the continuous improvement tool. Examples include but are not limited to: (1) investigation of Sentinel or Never events, (2) review of patient outcomes from previous cases with reviewer defined criteria, (3) monitoring the frequency of reviewer defined criteria, (4) assessing compliance to patient care protocol requirements, (5) alerting the reviewer of new cases meeting the defined criteria, etc.

Medical Facilities have an obligation to implement a continuous improvement program focused on improving patient outcomes. Therefore, the continuous improvement tools supports the medical facility's continuous improvement plan. While there are many reasons for using the continuous improvement tool, the following use case is provided to demonstrate how one reason may leverage the capabilities of the continuous improvement tool:
When Sentinel or Never Events occur the requirements are (1) to review the specific conditions leading up to the event and where possible take the appropriate actions to prevent recurrence. This includes a failure investigation focused on identifying the root cause(s) of the event. It is not uncommon to identify several potential root cause(s). Therefore, effective failure investigation requires the ability to assess the impact of all patient interaction (patient care activities and conditions) and the patient's response. However, currently electronic patient records are limited to a series of snap shots in time during patient care. The snap shots routinely do not provide an effective means to assess the patient interactions and response. Therefore, the determination of correlation between any specific patient interaction may be limited by the lack of some number of scientifically significant facts required to make the determination.

The inability to effectively implement process controls is a significant limitation of both paper and Electronic Health Records (EHR). The patient records limitations include the information included in the patient record does not provide enough details to clearly assess the relevant information available to the practitioner at the time of the event or prior to the event. The limitations could include, for example, the numeric values of patient vital signs, the waveform information, the specific time lines for interactions, and access to data not contained in the patient record

Numeric values of patient vital signs displayed on medical device screens are not collected frequently enough to properly assess the patient response to a specific interaction with the patient. Waveform information displayed on medical devices may not be part of the patient record. Even when present, the content is a series of snap shots at prescribed intervals. The snap shots do not routinely provide the details needed. Specific time lines do not show all patient interactions with patient and the vital signs before and after each patient interaction, if they exist. Accessing past data is limited to the data contained in the patient record. Ability to access any other historical data is limited, if not impossible, if the historical data is not contained in the patient record. Since, for the most part, the current systems do not collect the data, it is not available for subsequent review.

Once the root cause(s) have been identified the process requires the identification of potential corrective/preventive actions that will eliminate or at least substantially eliminate the root cause(s). To accomplish this the potential-actions need to be assessed and prior to implementation verify and validate that the actions are effective in eliminating/substantially reducing the root causes, without creating new potential issues.

The Medical Facilities are looking to eliminate unexpected outcomes and ensure expected outcomes (e.g. patients receive all scheduled anti emetics or multi modlas). Improved patient outcome requires a continuous spiral of improved process control. Therefore, continuous improvement programs focus of identifying opportunities to eliminate unexpected outcomes. The program looks for situations where the existing process controls or procedures yield negative and/or unexpected outcomes. Truly advanced programs also look for situations where the outcomes are more positive than expected. When situations presenting opportunities for improvement are identified, the organization determines the risk of a repeat event and prioritizes resources to address the top opportunities. The identified situations are tracked and managed in the Corrective and Preventive (C/P) Action process. This process requires documented Failure Investigation details, assessment of potential corrective and/or preventive actions, and verification and validation outcomes of the C/P actions tried. The current systems do not adequately support an effective Continuous Improvement Program.

The tools currently available are limited with respect their ability to support traditional failure investigation, process control methodology and verification and validation of proposed corrective and preventive actions.

Acquisition of data from multiple disparate sources, consolidation of all information within a unified view, running process controls and/or workflows and delivering actionable insights to specific users in near-real time has been a problem.

For example, U.S. Pat. No. 7,315,825 to Rosenfeld et al teaches a rules-based patient care system for use in remote monitoring healthcare locations with the purpose of supporting telemedicine during patient treatment. A patient rules generator creates rules for the patients. The rules generator acquires performance measures indicative of the ability of a rule to predict changes in the condition of the patient. A determination is made from the rules performance measures whether to revise the rule. A rules engine applies a rule to selected data elements stored in the database to produce an output indicative of a change in the medical condition of the patient. The output from the rules engine is used to determine if intervention is warranted. But, the remote monitoring only involves a current review. It does not allow for a retrospective view.

To fully support telemedicine and after the fact assessments of specific patient care procedures; there is a real need to be able to have access to all the relevant during the patient care. This supports the assessment of the patient responses to various potential contributors during the patient care. Without the ability to select specific times during the patient care to assess the entire history of the patient care, the assessments are limited to an incomplete picture of the whole story.

### SUMMARY OF THE INVENTION

The continuous improvement tool of the present invention is designed to facilitate the analysis of data and the impact of potential changes to the protocol. The continuous improvement tool is designed to interact with a process control system capable of collecting data from various sources including electronic sources including medical devices, lab records, patient care records, image records, etc. The process control system stores not only the data required for the patient care records but also any number of parameters identified as quality data.

The continuous improvement tool of the present invention allows analysis of historical and future cases meeting the criteria defined by the potential actions to be considered. Therefore, the tool provides a means to effectively assess the impact of the changes. Without the tool a commonly accepted process control would be to use design of experiment tools in controlled studies. With the tools the required analysis rapidly provides knowledge available from historical cases. Additionally, if any new cases meeting the criteria the reviewer may be notified of the new case.

The continuous improvement tool of the present invention provides means to (1) access and assess the data for specific cases, (3) access and assess other cases with the reviewer's identified and defined conditions from historical records or future records as they occur. The availability of process control tools has not been available to Medical Facilities. These tools have the ability to collect and store data from any source.

Thus, the present invention is directed to a continuous improvement system and method to operationalize process controls for medical patients to improve patient outcomes and includes a telecommunications network; at least one monitoring station comprising monitoring equipment where the monitoring equipment includes instructions for monitoring data elements and for sending the monitored data elements via the telecommunications network, and includes instructions for receiving monitored data elements from patients and accessing patient data elements indicative of a medical conditions associated with each of the patients; a patient database containing information concerning the medical condition, history, and status of each of the patients; at least one communication hub comprising instructions for collecting data from any number of electronic devices including medical devices and instructions for transmitting data to any number of electronic devices including medical devices, as well as instructions for storing data associated with the patient records and/or data to be stored as quality data; a data storage engine comprising a means for collecting data from any number of electronic devices including medical devices and instructions for transmitting data to any number of electronic devices including medical devices, as well as instructions for storing data associated with the patient records and/or data to be stored as quality data; the telecommunication network providing access to all data, including continuous wave form data, collected during the treatment of the patient and quality data not included in any patient record storage location; and a user interface rules engine that provides the user with ability to select any point in time during the patient treatment to:
i. review the details collected regarding the treatment at the selected time
ii. review the details before or after the selected time.
iii. create guidance rules to identify cases identified as complying with the defined rules, the user may define the period of time used to identify the cases for review, the user may select future cases only, or past cases to some defined date, or a combination of the two.
iv. define who, when and how to communicate that cases meeting the defined criteria are available for review, in this case the who may be only the individual(s) evaluating the defined criteria without notification to anyone monitoring a current case, or including specific individuals monitoring the current case;

where the user interface rules engine includes:
means to collect, store and process data in near real-time,
means to compose views that organize data for end-users to consume,
means to let a user create execution steps on the data streams,
means to notify end-users based on execution steps defined by end-users,
means to display an organized view of data within a timeline of events,
means for end-users to change or augment the execution steps, and

means to provide notifications at the same time the end-user is reviewing data,

so that data from multiple disparate sources can be acquired, consolidated within a unified view, process controls and workflows can be run, and actionable insights can be delivered to specific users in near-real time.

### DESCRIPTION OF THE DRAWINGS

The foregoing and other features and advantages of the present invention will become apparent to those skilled in the art to which the present invention relates upon reading the following description with reference to the accompanying drawings, in which:

FIG. 1 is a flow diagram of a global view of an Interoperability Environment showing the various communication sources and targets;

FIG. 2 is a flow diagram of a global view of the continuous improvement system of the present invention;

FIG. 3 is a screen shot showing a case analysis review screen;

FIG. 4 is a screen shot showing a case analysis review screen showing interaction with data options;

FIG. 5 is a screen shot showing a case analysis review screen which includes waveform displays;

FIG. 6 is a screen shot showing a case analysis review screen showing a guidance tool to create or modify a guidance;

FIG. 7 is a screen shot showing a case analysis review screen showing the selection of a primary filter;

FIG. 8 is a screen shot showing a case analysis review screen indicating that the event is an incision;

FIG. 9 is a screen shot showing a case analysis review screen which provides an airway summary;

FIG. 10 is a screen shot showing a case analysis review screen showing that the anesthesia is general, as well as the secondary and third level filters; and

FIG. 11 is a screen shot showing a case analysis review screen which defines the scope of the fourth level filter.

### DETAILED DESCRIPTION OF THE INVENTION

The present invention is directed to a continuous improvement system and method for medical patients to improve patient outcomes which includes a telecommunications network; at least one monitoring station comprising monitoring equipment where the monitoring equipment includes instructions for monitoring data elements and for sending the monitored data elements via the telecommunications network, and includes instructions for receiving monitored data elements from patients and accessing patient data elements indicative of a medical conditions associated with each of the patients; a patient database containing information concerning the medical condition, history, and status of each of the patients; at least one communication hub comprising instructions for collecting data from any number of electronic devices including medical devices and instructions for transmitting data to any number of electronic devices including medical devices, as well as instructions for storing data associated with the patient records and/or data to be stored as quality data; a data storage engine comprising a means for collecting data from any number of electronic devices including medical devices and instructions for transmitting data to any number of electronic devices including medical devices, as well as instructions for storing data associated with the patient records and/or data to be stored as quality data; the telecommunication network providing access to all data, including continuous wave form data, collected during the treatment of the patient and quality data not included in any patient record storage location; and a user interface rules engine that provides the user with ability to select any point in time during the patient treatment to:
i. review the details collected regarding the treatment at the selected time
ii. review the details before or after the selected time.
iii. create guidance rules to identify cases identified as complying with the defined rules, the user may define the period of time used to identify the cases for review, the user may select future cases only, or past cases to some defined date, or a combination of the two.
iv. define who, when and how to communicate that cases meeting the defined criteria are available for review, in this case the who may be only the individual(s) evaluating the defined criteria without notification to anyone monitoring a current case, or including specific individuals monitoring the current case;

where the user interface rules engine includes:
means to collect, store and process data in near real-time,
means to compose views that organize data for end-users to consume,
means to let a user create execution steps on the data streams,
means to notify end-users based on execution steps defined by end-users,
means to display an organized view of data within a timeline of events,
means for end-users to change or augment the execution steps, and

means to provide notifications at the same time the end-user is reviewing data,

so that data from multiple disparate sources can be acquired, consolidated within a unified view, process controls and workflows can be run, and actionable insights can be delivered to specific users in near-real time.

The present invention allows for the acquisition of data from multiple disparate sources, consolidation of all information within a unified view, running process controls/workflows and delivering actionable insights to specific users in near-real time. The present system identifies which information is actionable based on user criteria using decision support algorithms and management software. Actionable information can be presented with rich context compared to typical isolated ancillary systems. The information can be personalized to patient, clinical and/or admin user. Users can replay the timeline of events with all relevant information under a given context. Information can be added or deleted to sharpen context, to gain additional knowledge, or to immediately implement changes based on the review.

The novel process improvement tool enables collection, storage and automation of process controls and/or workflows on vast disparate data streams that normally are not accessible with paper records or poorly accessible due to isolated ancillary data systems with current electronic systems. Workflow automation changes and data points collected can be easily augmented. The improvement tool of the present invention gives users unique ability to dynamically organize data into views of patient populations regardless of geographic location, with additional ability to pare view according to user specified criteria and the ability to collect data from devices, HIT systems, external sources, user input, or any other source of data which can be made available in electronic format.

The data entry can be a mix of automated and user input. The present invention has the ability to use any of the above to create a set of evaluations on the data stream to trigger notifications intended to notify about deviations from expected workflow, process control or clinical course, as well as the ability to review and replay the sequence of data points in the past so that users can engage in a critical evaluation of a specific event or sequence of events that led to a negative clinical outcome, or non-compliance with or failure of a process control or workflow.

The present invention has the ability to use historical data to generate guidances to manage clinical conditions or new process controls/workflows in real time and the ability for a user to acknowledge that a clinical guidance was true/valid in real time.

The present invention has the ability to change execution pathway per user criteria depending on inputs in real time (e.g. data from a micro assessment could change the frequency of future assessments etc.), as well as enable end-users who are consuming the notifications of the improvement tool to direct and coordinate the team to change the input provided to the improvement tool at the time of the review of data so that the any updated workflow, for instance with additional evaluations, or modified evaluations.

The present invention has the ability to collect, store, and process data in near real-time. It can compose views that organize data for end-users to consume, to let the user create execution steps on the data streams, to notify end-users based on execution steps defined by end-users, and to present an organized view of data within a timeline of events. Further, the present invention has the ability for end-users to change or augment the execution steps and notifications at the same time the end-user is reviewing data.

The interface rules engine creates guidance rules one the end-user defines the criteria to be evaluated and the notifications that need to be delivered to the care team. End-user can define criteria based on any data-point available in the collected data stream, e.g., from medical devices. Users can be clustered into groups. Patients can be clustered into groups. Patients can be “tagged” with user-defined criteria. Patients can be stratified according to user-defined criteria in real time. An end user on the fly can alter certain thresholds.

When and how communications are made regarding cases meeting defined criteria that need review occur when authorized End-users who use the “continuous improvement tool” have the ability to define the communication type (for instance, email notification to Cockpit user, sms, and who receives it. Also, when they receive it. Communication will be delivered on any device as prescribed by authorized end user in order to optimally support defined workflow. End-user also can configure the escalation process to execute if the notification is not acknowledged or addressed. End users will have ability to “snooze” certain communications if allowed by authorized end user.

Improvement is achieved every time end-users realize that there is a difference between the process followed by end-users and a better process that they could have followed based on best practice, peer-reviews publications or reviews of data collected and stored by the continuous improvement tool. But, this is complex. Errors and negative events will be identified dynamically. When this happens retrospectively, all data will be available (including waveforms) to enable the richest possible clinical review. New guidances can be created in real time if specific sentinel events or sequence of data are identified. Deviations form an expected process or workflow can be identified in real time. Following evaluation, any changes to a process control or workflow can be implemented dynamically

The following terms used in the description that follows. The definitions are provided for clarity of understanding:

Assessment data is all data relevant to the health of a patient.

A “healthcare location” is a facility, whether temporary or permanent, that is not generally equipped to provide expert medical care on a twenty-four basis. By way of illustration and not as a limitation, a healthcare location may be a remote clinic, a doctor's office, a field hospital, a disaster aid station, a patient transport vehicle and similar care facilities

A Caregiver is an individual providing care to a patient. Examples include a nurse, a doctor, medical specialist (for example and without limitation an intensivist, cardiologist or other similar medical specialist).

Clinical data is data relating to the observed symptoms of a medical condition.

A Monitored patient is a person admitted to a healthcare location.

Monitored data is data received from monitoring devices connected to a monitored patient from whom monitored data is collected and whose condition is subject to continuous real-time assessment from a remote command center.

Patient data is data relating to a patient's diagnosis, prescriptions, history, condition, laboratory results and other health-relevant data.

Physiological data is any data relating to the functions of the human body and its processes. symptom—any sign or indication of a health condition that can be identified from patient reports and/or assessment data.

The present invention is directed to a continuous improvement system for medical patients which includes a telecommunications network, comprising at least one ECO system (as used herein, “ECO system,” “eco system”, and “ecosystem” can be used interchangeably) (communication hub). comprising instructions for collecting data from any number of electronic devices including medical devices and instructions for transmitting data to any number of electronic devices including medical devices, as well as instructions for storing data associated with the patient records and/or data to be stored as quality data, a patient database containing information concerning the medical condition, history, and status of each of the patients, a data storage engine comprising a means for collecting data from any number of electronic devices including medical devices and instructions for transmitting data to any number of electronic devices including medical devices, as well as instructions for storing data associated with the patient records and/or data to be stored as quality data, at least one communication hub comprising instructions for collecting data from any number of electronic devices including medical devices and instructions for transmitting data to any number of electronic devices including medical devices, as well as instructions for storing data associated with the patient records and/or data to be stored as quality data, where the telecommunication network providing access to all data, including continuous wave form data, collected during the treatment of the patient including quality data not included in any patient record storage location, and a user interface rules engine that provides the user with ability to select any point in time during the patient treatment to facilitate review the details collected regarding the treatment that the selected time, review the details before or after the selected time, creating guidance rules to identify cases identified as complying with the defined rules, the user may define the period of time used to identify the cases for review, the user may select future cases only, or past cases to some defined date, or a combination of the two, and to define who, when and how to communicate that cases meeting the defined criteria are available for review, in this case the who may be only the individual(s) evaluating the defined criteria without notification to anyone monitoring a current case, or including specific individuals monitoring the current case.

The present invention uses a telecommunications network to facilitate rules-based care of patients receiving care in a healthcare location. As used herein, a healthcare location may be a remote clinic, a doctor's office, a field hospital, a disaster aid station, a patient transport vehicle and similar care facilities. A patient may be selected for monitoring based on criteria established by the treatment facility. By way of illustration and not as a limitation, a “monitored patient” comprises a critically ill patient, an acutely ill patient, a patient with a specific illness, a patient with serious injuries, and a patient with an uncertain diagnosis.

An ECO system communication hub acquires monitored data elements from any electronic device including medical devices monitoring and/or treating a patient and transmits the monitoring data over a network to a storage location to be processed by the interoperability environment engine. Monitored data comprises physiological data elements, video data elements, and audio data elements. The remote command center receives the monitoring data from all patient monitoring stations. The interoperability environment engine also accesses other data relating to the condition of a patient. By way of illustration and not as limitation, the remote command center has access to data relating to personal information about the patient (name, address, marital status, age, gender, ethnicity, next of kin), medical history (illnesses, injuries, surgeries, allergies, medications), admissions information (symptoms, physiological data, time of admission, observations of admitting caregiver), treatment, lab data, test reports (radiology reports and microbiology reports for example), physician's notes, a patient's diagnosis, prescriptions, history, condition, laboratory results and other health-relevant data (collectively “patient data”) to the extent available from the healthcare location.

In the present invention, a monitored patient care system provides care to monitored patients based on the capabilities of the healthcare location. The rules engine, the decision support algorithms, the order writing software facilities, and the continued care software are adapted to the capabilities of the healthcare location based on the application of site assessment rules to the healthcare location. In the present invention, components of a healthcare location patient care system may be supplied to the healthcare location to improve the level of its treatment capabilities.

The present invention operates in the context of an Interoperability Environment which includes a hardware eco system comprising a rules engine to collect, translate, store and send electronic data to the core software engine for any electronic source via communication methods, a core software engine having means to collect and transfer electronic data from any number of sources including medical devices, clinical information systems, hospital information systems, a rules engine to apply rules to improve compliance with hospital approved protocols, standards and guidances, a rules engine to apply rules to update all subsystems using any given parameter when the parameter is updated in the official recognized source of truth for that parameter, a rules engine to apply rules to populate the computer information system (or CIS) with all required patient information, a rules engine to apply the rules to maintain all quality and process control data in a format supporting advanced analytics separate from the CIS data, a rules engine to provide a means to communicate notifications to any number of remote electronic devices without limitation of platform, rules engines to support data analysis including failure investigation and process control and machine learning tools, rules engines providing a means to improve adherence to medical practitioner directed patient care, and rules engine to provide two-way communication with electronic devices including medical devices.

The ECO system hardware acquires data elements from medical devices including patient monitors and transmits the data over a network to data storage, identified targets, individuals requiring notification, remote dashboards, remote mobile communication devices, remote monitoring locations. The collected data comprises physiological data elements, video data elements, audio data elements, manually entered patient evaluations, drug delivery, incision time and location, blood product delivery, lab results, admitting evaluation results, post-operative assessments, I/O data, etc. The Interoperability Environment also accesses other data relating to the condition of a patient. By way of illustration and not as limitation, the Interoperability Environment has access to data relating to personal information about the patient, medical history (illnesses, injuries, surgeries, allergies, medications, etc.), admissions information (symptoms, physiological data, time of admission, observations of admitting caregiver), treatment, lab data, test reports (radiology reports and microbiology reports for example), physician's notes, a patient's diagnosis, prescriptions, history, condition, laboratory results and other health-relevant data (collectively “patient data”) to the extent available from the healthcare IT network. The data collected over the network, that is, the monitoring data and the patient data, is collectively referred to as “assessment data.”

A preferred system utilizes an agnostic approach to communications between data sources and data targets. This allows the hospital to utilize any number of suppliers and/or device models within the interoperability environment. This eliminates the need to buy new equipment just to achieve interoperability. The system will allow access to all relevant patient data from all applicable sources as discussed above and a means to store accurately, timely and completely all relevant data regarding patient care and patient response/outcome.

The commonly accepted approach focuses on identifying the means to allow the patient monitoring medical devices to communicate with the medical devices providing patient therapy to appropriately adjust the therapy being provided. Current systems assume that only vital signs collected at the point of care are required to provide clinically proven algorisms all the information required to make the appropriate decision to adjust the medical device provided therapy, which limits the full potential of a device interoperability infrastructure. The present invention expands the capabilities of the system to include all available, but not limited to, medical devices, patient specific details that are applicable to developing and controlling the optimum patient care outcome.

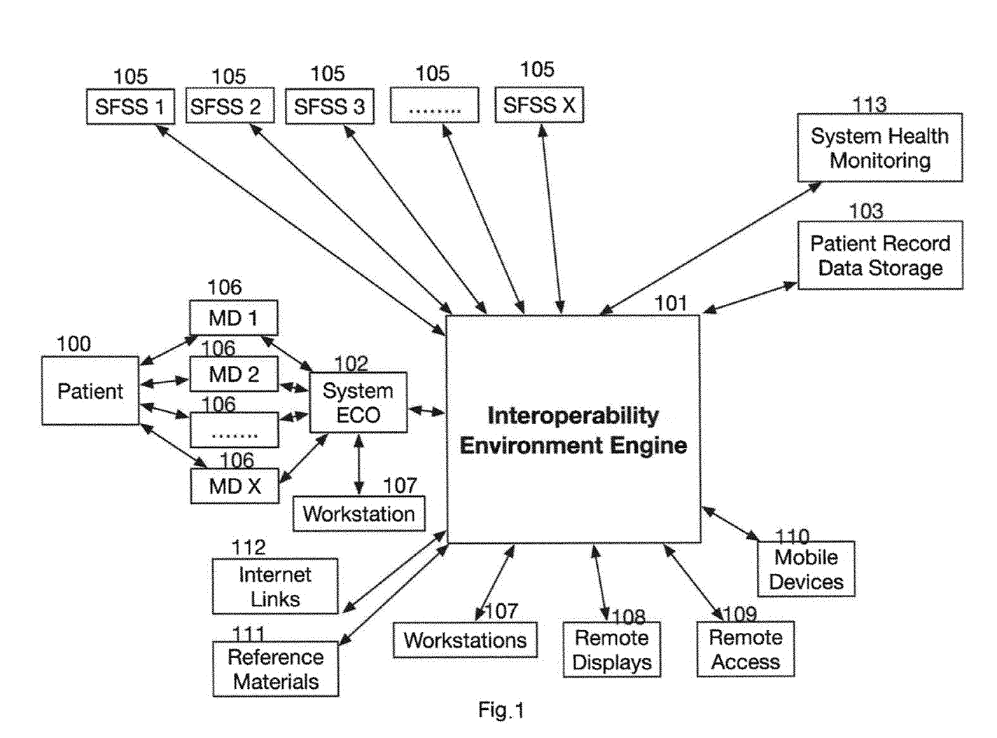

An interoperability environment is represented in FIG. 1, which is a block diagram of the global Interoperability Environment. At the center of FIG. 1 is the Interoperability Environment Engine 101, referred to as the engine. This is to symbolize that it is the core communication tool to ensure timely and accurate communication between the various sources and targets of the data being collected and shared in the global environment. As noted by the two-way arrows, the present invention also supports communication between any number of locations with connections to the Interoperability engine.

The Interoperability Environment is also composed of an ECO system 102. The ECO System is composed of any number of hardware options, utilizing various operating systems such as Linux, Windows, and MacOS. The ECO system resides in close proximity of the electronic devices, including electronic medical devices (or EMD) 106, talking with the ECO System, via any number of communication channels including LAN, Serial, Wi Fi, wireless, etc. The ECO system is utilized as the conduit between the medical devices and the engine to collect, translate and transfer electronic data to the engine for processing to the proper storage locations or specific data targets.

The Interoperability Environment is also composed of one or more data repositories to store all data collected prior to sending the data to any target location. For example, the data can come from the patient 100, electronic medical devices 102, work stations 103, patient record data storage 106, remote displays 108, remote access devices 109, mobile devices 110, reference materials 111, internet links 112, and the like. The engine tracks each data field based on a start date and end date of the parameter being collected. When combined with the engine time stamping and data collecting, the Interoperability Environment is capable of supporting data analysis of individual parameters as well as interactions with other parameters. Patient Record Data Storage 103 contains all required patient care data collected and stored by the engine.

Patient monitoring equipment acquires monitored data elements from a patient monitoring station and transmits the monitored data (sometimes also referred to herein as, “monitoring data”) over a network to a remote command center. Monitored data comprises physiological data elements, video data elements, and audio data elements. The remote command center receives the monitored data from all patient monitoring stations. The remote command center also accesses other data relating to the condition of a patient. By way of illustration and not as limitation, the remote command center has access to data relating to personal information about the patient (name, address, marital status, age, gender, ethnicity, next of kin), medical history (illnesses, injuries, surgeries, allergies, medications), admissions information (symptoms, physiological data, time of admission, observations of admitting caregiver), treatment, lab data, test reports (radiology reports and microbiology reports for example), physician's notes, a patient's diagnosis, prescriptions, history, condition, laboratory results and other health-relevant data (collectively “patient data”) to the extent available from the healthcare location. The data available to the remote command center over the network, that is, the monitored data and the patient data, is collectively referred to as “assessment data.”

A rules engine applies a rule or rule set to the data elements selected from the assessment data from each monitored patient to determine whether the rule for that site has been contravened. In the event the rule has been contravened, an alert at the remote command center is triggered. Rules for each monitored patient may be established and changed at the remote command center for each as the patients' conditions warrant. In one embodiment of the present invention, a rule is established to determine whether a patient's condition is deteriorating. An alert that a rule has been contravened comprises advice on treatment of the patient.

A patient rules generator establishes one or more rules for the monitored patient associated with a patient monitoring station. The patient rules generator collects rules performance measures indicative of the ability of the rule to predict changes in the condition of a patient and uses these measures to assess the efficacy of the rule. The patient rules generator may update a rule, determine that a rule is acceptable as is, or determine that there is insufficient data to revise a rule.

The patient rules generator may also evaluate the assessment data of patients with similar conditions to determine whether a predictive rule can be written and applied to patients with the same or similar conditions. The patient rules generator may also test a proposed rule against historical data to determine whether the rule is predictive of a change in a patient's condition. The patient rules generator generates a rule that is consistent with the service level measures established by a site assessment module.

The present invention provides continued care software that uses elements of the assessment data to provide decision support and that prompts a user for input to provide decision support to caregivers. A decision support algorithm responds to elements of assessment data to produce textural material describing a medical condition, scientific treatments and possible complications. This information is available in real time to assist in all types of clinical decisions from diagnosis to treatment to triage.

In the present invention, a healthcare location patient care system provides care to healthcare location patients based on the capabilities of the healthcare location. In this embodiment, the rules engine, the decision support algorithms, the order writing software facilities, and the continued care software are adapted to the capabilities of the healthcare location based on the application of site assessment rules to the healthcare location. Components of a healthcare location patient care system may be supplied to the healthcare location to improve the level of its treatment capabilities. In still another embodiment of the present invention, components of the healthcare location are packaged and assigned a site assessment code. The code is used by the remote command center to predetermine elements of the site assessment process thereby simplifying that process.

In the present invention, patient-monitoring equipment acquires monitored data elements from a patient monitoring station and stores monitoring data locally. The stored monitoring data is sent to a remote command center along with patient data at a pre-established time or when requested by remote command center. The remote command center evaluates the “delay” monitored data and assessment data in the same manner as if these data were received in real time. By way of illustration, the remote command center will apply the rules engine and the decision support algorithms to the delayed monitored data and patient data and provide guidance to the healthcare location. The present invention thus provides high quality care in environments where continuous high bandwidth communications are not available or economically infeasible.

The system of the present invention collects the data and stores the data. The patient record in the medical facility electronic medical record system (EMR) contains the same information as is known in the art with other systems. The EMR is one target and receives only the data required by the specific target. The data sent to the EMR plus any other data collected is stored in the cloud and filed to support the assessment of the case. There is the ability to store some of the data as case record and other additional data collected in the Quality data. Thus, the present invention can add functionality and features by finding ways to more fully leverage the knowledge that is gained as the system gains knowledge. Thus the system has the ability to grow as new knowledge is gained.

The invention is capable of collecting any identified data at a frequency that supports a meaningful assessment of patient interactions and patient response to those interactions. The term data includes but is not limited to patient medical history, patient interaction details including person performing the interaction, the time provided, any drug, disposable or medical device used to complete the interaction, numeric patient vital signs provided by active patient monitors, Wave forms provided by active patient monitors, changes to settings of any medical device used on the patient, lab results, practitioner notes and documented observations.

The invention is capable of displaying the information along a time line of the treatment period. The display may be configured to provide a graphical representation of any numeric patient vital sign collected during the treatment. Additionally, the invention supports the viewing of the waveform data generated during the treatment as an accurate representation of the waveform screens on the active patient monitors.

Using the review screen the user may select any specific time on the time line to review the patient vital signs values at that time. With respect to numeric and wave form data the system has the ability to move forward and backward during the treatment to assess potential link between patient interaction and patient response.

Using these tools, the person or team conducting the assessment may identify potential root causes and or potential corrective/preventive actions including the establishment of guidances. The system is designed to allow the user to establish parameter or conditional limits to implement guidances. In this case implement implies that the system will monitor future treatments (same treatment as reviewed) against specified conditions and when any of those conditions are observed, an alert/notification will be sent. The level of implementation may be limited to monitoring in the back ground without any interaction with the provider. In this case the individual(s) identified by the guidance will be notified that a treatment case of interest is available for review. Note: It is possible for several guidances to be created and implemented to accelerate the collection of data for various alternative approaches. Using this approach, the data necessary to build the required scientific evidence to justify the appropriate treatment protocol guidance may be collected without any change to currently approved protocols.

Once the scientific evidence has been collected to justify a formal change to the Medical Facility's protocol, the appropriate review can be performed and if adequately justified, approved for implantation via changes to the authorized protocol. As knowledge is gained using this basic design of experiment methodology, sufficient logic may be developed to support the use of machine learning tools to further accelerate the continuous improvement program.

The present invention supports the ability to move back in time during the current patient care under review. The same screens may also be utilized to evaluate the case during formal reviews. These screens also include icons to show when specific patient interactions were performed.

The waveform data is collected directly from the medical devices. These wave forms are stored with the patient records. Once stored the waveforms may be displayed and played. With the ability to play the stored wave forms the system is also includes to play, replay, fast forward or fast reverse to identify the time periods of most interest to the medical practitioner reviewing these records. Continuous improvement program focused on patient outcome:

Improved patient outcome requires a continuous spiral of improved process control. Therefore, continuous improvement programs focus of identifying opportunities to eliminate unexpected outcomes.

The present invention looks for situations where the existing process controls or procedures yield negative outcomes. Truly advanced programs also look for situations where the outcomes are more positive than expected. When situations presenting opportunities for improvement are identified, the organization determines the risk of a repeat event and prioritizes resources to address the top opportunities. The identified situations are tracked and managed in the Corrective and Preventive Action process. This process requires documented Failure Investigation details, assessment of potential corrective and/or preventive actions, verification and validation outcomes of the C/P actions tried. As discussed above, the current prior art systems do not adequately support an effective Continuous Improvement Program.

### Example of the Continuous Improvement Tool

The system of the present invention will best be used when there is a specific reason to analyze the details of a case. For example, in response to a Sentinel or Never event. However, the tool can be used for detailed analysis of any historical data. As customer knowledge is gained by using various analytical tools, the tool can be expanded to assess historical records which meet the criteria selected for review. This historical review may be used to collected the required scientific justification to validate any proposed change.

FIG. 1 is a flow diagram which shows the continuous improvement tool utilization logic and process of the present invention. It begins by identifying the case of interest, accessing the case, and reviewing the case to identify items or periods of interest. After an iterative process of analysis, guidance rules are created and applied to cases of interest, including notifications and communications about protocol changes and implementation.

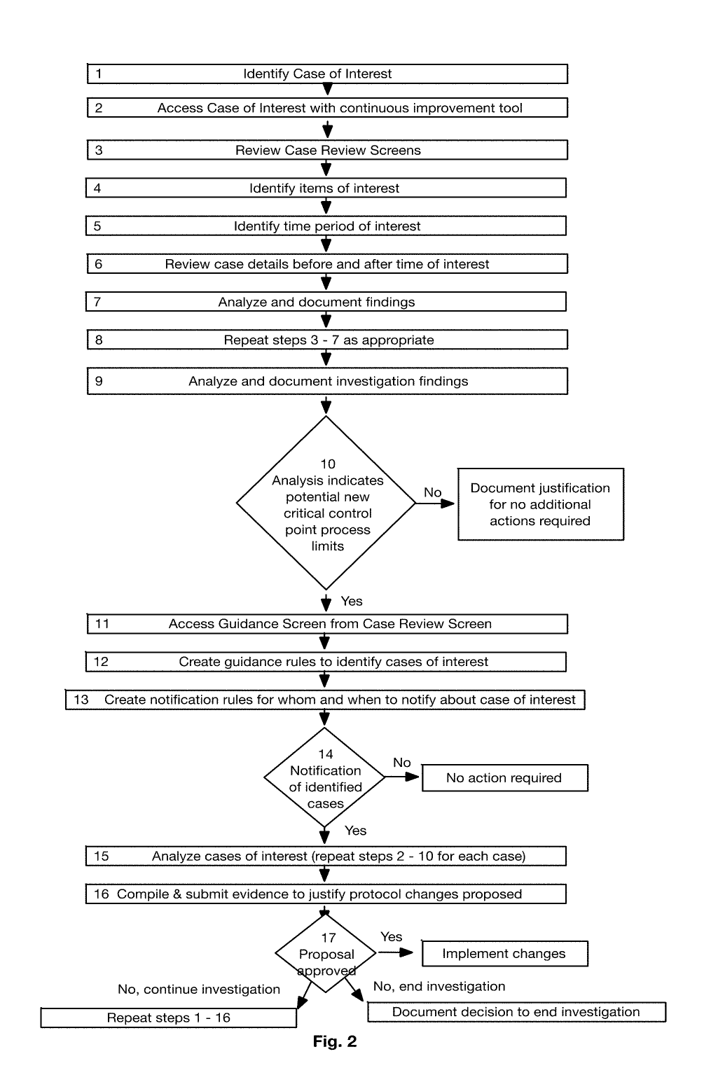

FIGS. 2-10 provide an example for an anesthesia protocol. However, the example and its slides and explanation of each screen are not intended to limit the scope of the capabilities of the Continuous Improvement Tool. The tool may be configured to support any number of processes.

In the present example of a case analysis, a review screen is provided that provides on overview of the case including, but not limited to:
Graphical representation of the patient vital signs during the case, including icons indicating when specific interaction with patient occur
   Legend of the graphs
   Drug delivery
   Incision
   Ventilation
   Case Detail Selection Keys
   Patient—details
   Case
   Data
   Guidance

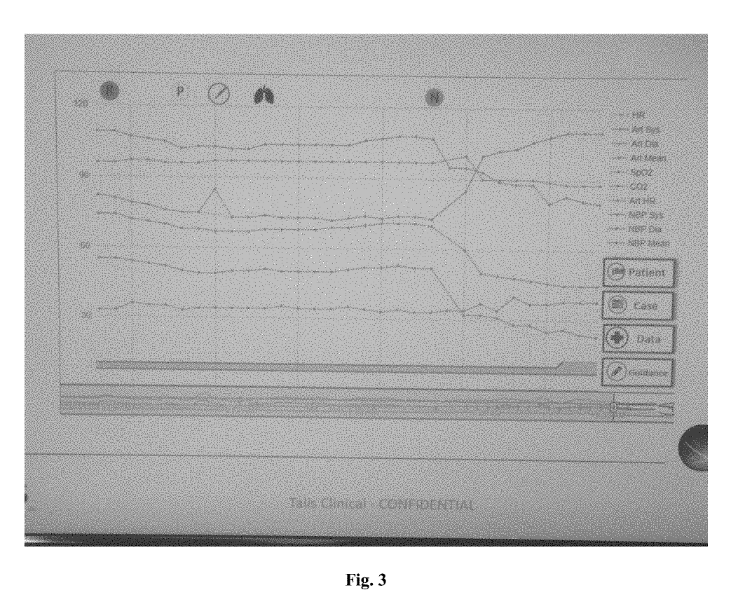

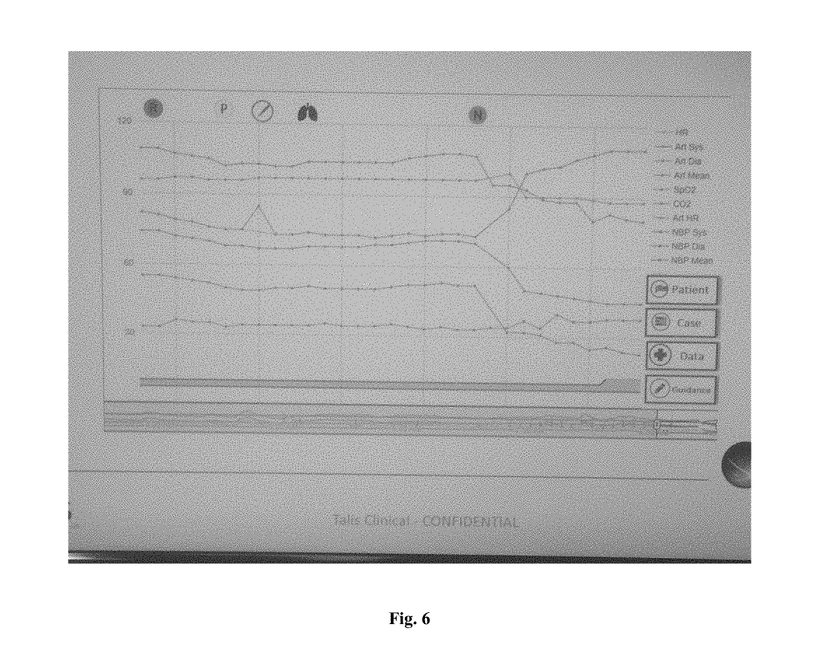

The screenshots provided a walk through of an example where data collected through a case is reviewed. In the timeline, data from multiple sources is visible along with clinical events, (i.e. incision) The screenshots, FIGS. 3,4, and 5 show how clinicians interactively review data along with the guidance editor. Then can evaluate their current process controls (FIG. 6) and make changes to processes based on the review of the case. The screenshots show an example of how this happens. For example, during a review clinicians might identify that the data showed in the screens of the review tool might be more effective if it also showed data from available, from cerebral oximetry. Because the platform support Cerebral monitors, clinicians can request to include cerebral oxymeters in the data set collected, so that clinicians can review and use its data to create more effective guidance.

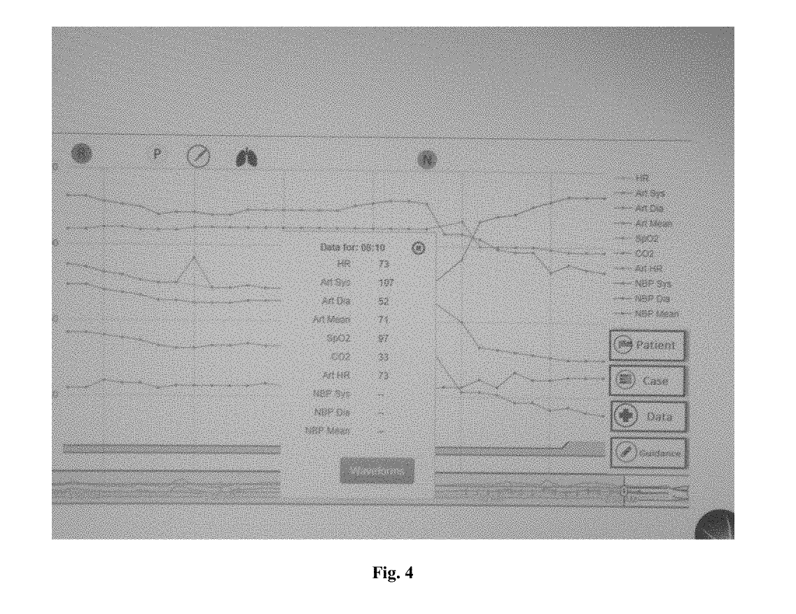

FIG. 3 is a screen shot of a review screen showing an interaction with the review screen data options, including an ability to move the cursor to a specific time in the case and clicks to see specific data details. This screen shot displays the actual collected data at the requested time of the case in the screen shot in FIG. 4.

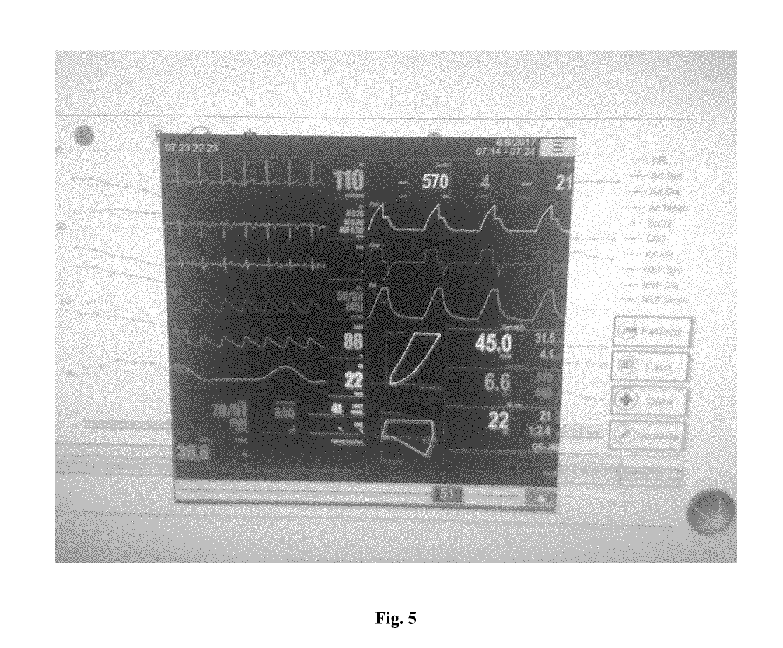

To see waveforms the user clicks on the waveform key, as shown in the screen shot in FIG. 3 and the waveform data collected is displayed as shown in the screen shot in FIG. 5

As seen in the screen shot is FIG. 6, there is a slide at the bottom of the waveform display, which enables the user to move the time back or forward to review changes before or after the selected time.

The screen shots illustrate how, from the review screen the user can access the guidance tool which walks the user through the process to create or modify a guidance.

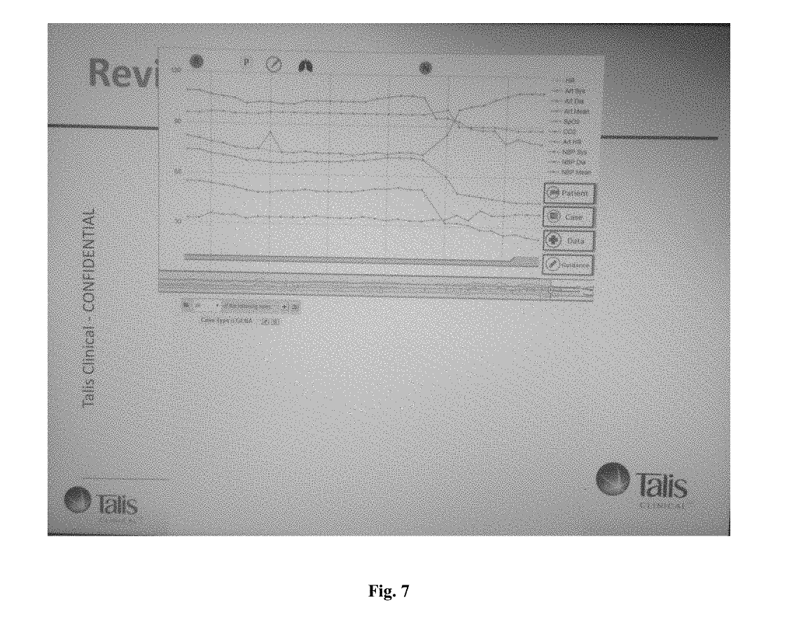

FIG. 7 shows the review screen and the selection of the scope of the primary filter which in this case defines the case type a GENA.

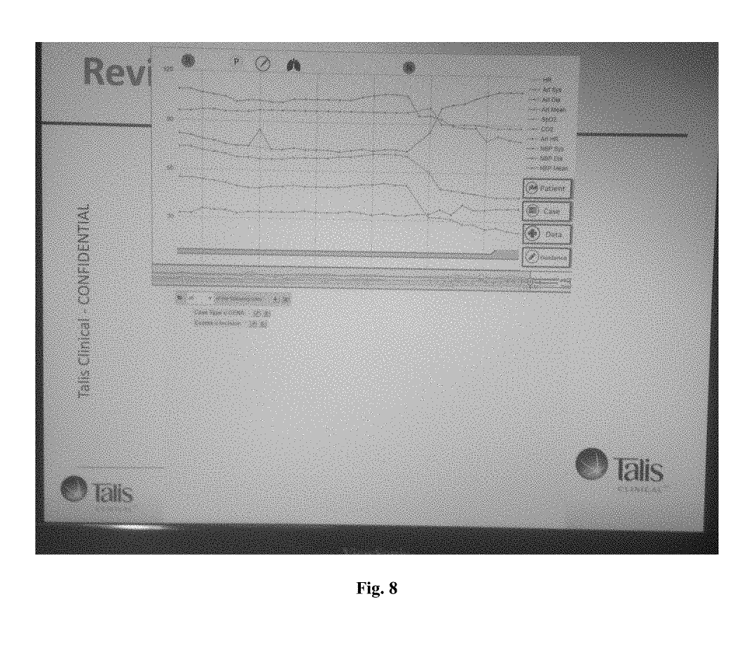

FIG. 8 shows the review screen and patient interactions, as well as showing that the event is an incision.

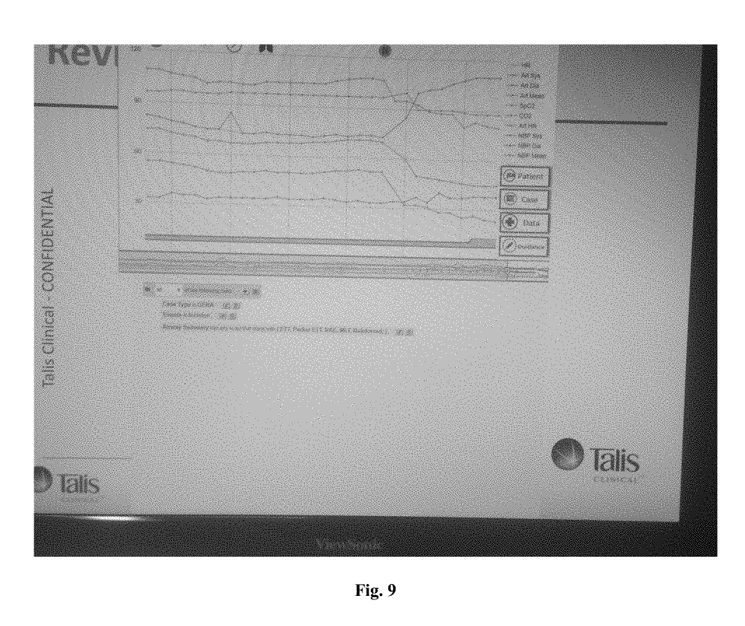

FIG. 9 shows a screen shot of an airway summary and has any list that starts with ETT, Parker ETT, RAE, MLT and/or reinforced.

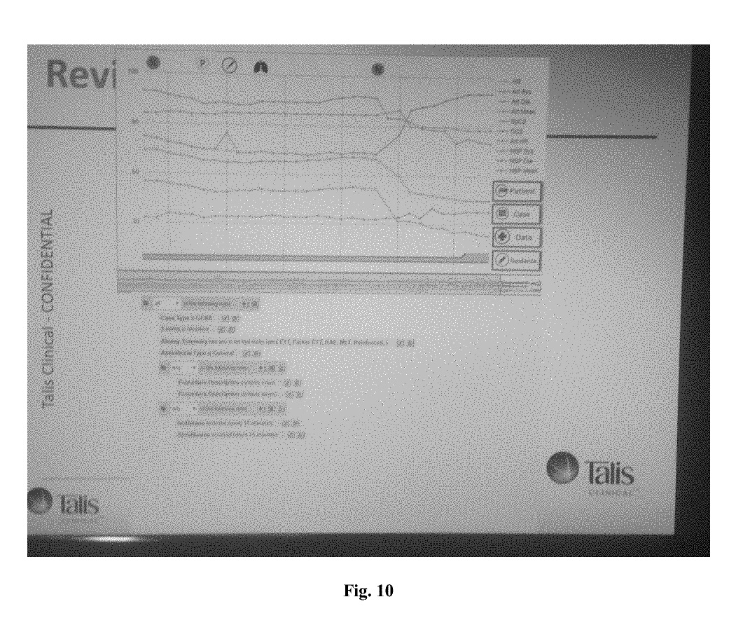

FIG. 10 shows a screen shot indicating that the anesthesia type is general and defines the scope of secondary filter according to certain rules, including whether the procedure description contains crani and/or the procedure description contains neuro. FIG. 10 also shows the definition of the 3rd level filter, and indicates that Isoflurane occurred before 15 minutes and Sevoflurane occurred before 15 minutes.

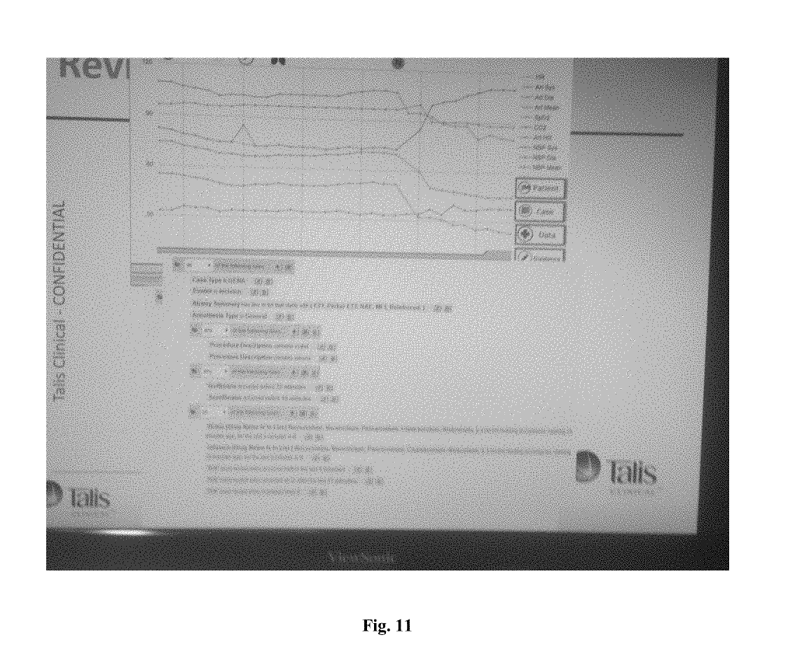

FIG. 11 shows the scope of a 4th level filter as defined by the following rules;
Bolus (Drug Name is in List (Rocuronium, Vecuronium, Pancuronium, Atracurium),) recent reading occurrences starting 10 minutes ago for the last 9 minutes is 0
Infusion (Drug Name is in List (Rocuronium, Vecuronium, Pancuronium, Atracurium),) recent reading occurrences starting 10 minutes ago for the last 9 minutes is 0
TOF most recent entry occurred before the last 5 minutes
TOF most recent entry occurred on or after the last 11 minutes
TOF most recent entry is greater than 2

The foregoing embodiments of the present invention have been presented for the purposes of illustration and description. These descriptions and embodiments are not intended to be exhaustive or to limit the invention to the precise form disclosed, and obviously many modifications and variations are possible in light of the above disclosure. The embodiments were chosen and described in order to best explain the principle of the invention and its practical applications to thereby enable others skilled in the art to best utilize the invention in its various embodiments and with various modifications as are suited to the particular use contemplated.

What we claim is:

1. A continuous improvement system for medical patients to improve patient outcomes comprising:
a telecommunications network;
at least one monitoring station comprising monitoring equipment wherein the monitoring equipment includes instructions for monitoring data elements and for sending the monitored data elements via the telecommunications network, and includes instructions for receiving monitored data elements from patients and accessing patient data elements indicative of a medical conditions associated with each of the patients;
a patient database containing information concerning the medical condition, history, and status of each of the patients;
at least one communication hub comprising instructions for collecting data from any number of electronic devices including medical devices and instructions for transmitting data to any number of electronic devices including medical devices, as well as instructions for storing data associated with the patient records and/or data to be stored as quality data;
a data storage engine comprising a means for collecting data from any number of electronic devices including medical devices and instructions for transmitting data to any number of electronic devices including medical devices, as well as instructions for storing data associated with the patient records and/or data to be stored as quality data;
said telecommunication network providing access to all data, including continuous wave form data, collected during the treatment of the patient and quality data not included in any patient record storage location; and
a user interface rules engine that provides the user with ability to select any point in time during the patient treatment to:
i. review the details collected regarding the treatment at the selected time
ii. review the details before or after the selected time.
iii. create guidance rules to identify cases identified as complying with the defined rules, the user may define the period of time used to identify the cases for review, the user may select future cases only, or past cases to some defined date, or a combination of the two.
iv. define who, when and how to communicate that cases meeting the defined criteria are available for review, in this case the who may be only the individual(s) evaluating the defined criteria without notification to anyone monitoring a current case, or including specific individuals monitoring the current case;
wherein said user interface rules engine includes:
means to collect, store and process data in near real-time,
means to compose views that organize data for end-users to consume,
means to let a user create execution steps on the data streams,
means to notify end-users based on execution steps defined by end-users,
means to display an organized view of data within a timeline of events,
means for end-users to change or augment the execution steps, and
means to provide notifications at the same time the end-user is reviewing data.
whereby data from multiple disparate sources can be acquired, consolidated within a unified view, process controls and workflows can be run, and actionable insights can be delivered to specific users in near-real time.

2. The system of claim 1 wherein the system identifies which information is actionable using an algorithm based upon user criteria.

3. The system of claim 1 wherein the system can replay the timeline of events with all relevant information under a given context and information can be added or deleted to sharpen context, to gain additional knowledge, and to immediately implement changes based upon the review.

4. A method of continuous improvement for medical patients to improve patient outcomes comprising:
providing a telecommunications network;
providing at least one monitoring station comprising monitoring equipment wherein the monitoring equipment includes instructions for monitoring data elements and for sending the monitored data elements via the telecommunications network, and includes instructions for receiving monitored data elements from patients and accessing patient data elements indicative of a medical conditions associated with each of the patients;
providing a patient database containing information concerning the medical condition, history, and status of each of the patients;
providing at least one communication hub comprising instructions for collecting data from any number of electronic devices including medical devices and instructions for transmitting data to any number of electronic devices including medical devices, as well as instructions for storing data associated with the patient records and/or data to be stored as quality data;
providing a data storage engine comprising a means for collecting data from any number of electronic devices including medical devices and instructions for transmitting data to any number of electronic devices including medical devices, as well as instructions for storing data associated with the patient records and/or data to be stored as quality data;
said telecommunication network providing access to all data, including continuous wave form data, collected during the treatment of the patient and quality data not included in any patient record storage location; and
providing a user interface rules engine that provides the user with ability to select any point in time during the patient treatment for:
i. reviewing the details collected regarding the treatment at the selected time
ii. reviewing the details before or after the selected time.
iii. creating guidance rules to identify cases identified as complying with the defined rules, the user may define the period of time used to identify the cases for review, the user may select future cases only, or past cases to some defined date, or a combination of the two.
iv. defining who, when and how to communicate that cases meeting the defined criteria are available for review, in this case the who may be only the individual(s) evaluating the defined criteria without notification to anyone monitoring a current case, or including specific individuals monitoring the current case;
wherein said user interface rules engine includes:
means to collect, store and process data in near real-time,
means to compose views that organize data for end-users to consume,
means to let a user create execution steps on the data streams,
means to notify end-users based on execution steps defined by end-users,
means to display an organized view of data within a timeline of events,
means for end-users to change or augment the execution steps, and
means to provide notifications at the same time the end-user is reviewing data.
whereby data from multiple disparate sources can be acquired, consolidated within a unified view, process controls and workflows can be run, and actionable insights can be delivered to specific users in near-real time.
